(19) United States
(12) Patent Application Publication
(10) Pub. No.: US 2026/0115389 A1
(43) Pub. Date: Apr. 30, 2026

(54) SYRINGE ADAPTER

(71) Applicant: Theodore Heierman, West Melbourne, FL (US)

(72) Inventor: Theodore Heierman, West Melbourne, FL (US)

(21) Appl. No.: 18/931,747

(22) Filed: Oct. 30, 2024

(51) Int. Cl. A61M 5/34; A61M 39/10

(52) U.S. Cl. CPC A61M 5/345; A61M 39/10; A61M 2039/1077

(57) ABSTRACT

An adapter for securing a tube having contents to a syringe including: (1) a conduit having a first end and an opposing second end; (2) a tube capture wall extending from the first end of the conduit, having threads located along a surface of the tube capture wall; (3) a second end wall having an inner surface defining the second end of the conduit and an opposing exterior surface; (4) a first protuberance extending from the exterior surface of the second end wall; and (5) a second protuberance extending from the exterior surface of the second end wall at a location opposing the first protuberance.

### FIELD OF THE INVENTION

The present invention relates to systems and methods for measuring amounts of product dispensed from a tube. More specifically, the present invention relates to a system for providing a connection between a tube having contents and a syringe.

### BACKGROUND OF THE INVENTION

Proper dosing of medications and other products is important for maintaining patient health and achieving efficacy of the administered medicine. Some medications and similar products are provided in tubes. Dispensing precise amounts of these products or measuring how much of the product is dispensed is often difficult. Therefore, a need exists for transferring a product stored in a tube into a device that can measure and administer the product without creating waste or contaminating the product.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to an adapter for securing a tube having contents to a syringe. The adapter may include a conduit, a tube capture wall, a second end wall, a first protuberance, and a second protuberance. The conduit may have a first end and an opposing second end. The tube capture wall may extend from the first end of the conduit. The second end wall may have an inner surface defining the second end of the conduit. The second end wall may also have an exterior surface opposing the inner surface. The first protuberance may extend from the exterior surface of the second end wall. The second protuberance may extend from the exterior surface of the second end wall at a location opposing the first protuberance.

Threads may be located along a surface of the tube capture wall. The threads may be located on an interior surface of the tube capture wall.

The tube capture wall may define a cylindrical volume having a diameter greater than an inner or outer diameter of the conduit. A longitudinal axis of the cylindrical volume may be parallel to a longitudinal axis of the conduit.

The system may include a planar surface located between the conduit and the tube capture wall. The planar surface may be perpendicular to the longitudinal axis of the conduit.

The system may further include an annular ridge located on the planar surface. The annular ridge may lie in a plane perpendicular to the longitudinal axis of the conduit. The annular ridge may protrude from the planar surface distal the conduit.

The second end wall may be adapted to secure to a luer lock syringe.

An exterior surface of the conduit may include a taper from a larger perimeter to a smaller perimeter.

An exterior surface of the conduit may include a plurality of ridges extending parallel to a longitudinal axis of the conduit. The plurality of ridges may extend continuously from a first end of the tube capture wall, which is distal the conduit, to a first point along the exterior surface of the conduit.

An outer circumference of the conduit proximate the first point may be larger than the outer circumference of the conduit at a second point along the exterior surface of the conduit. A first distance between the first point and the second end wall may be greater than a second distance between the second point and the second end wall.

An outer circumference of the conduit at a third point along the exterior surface of the conduit may be less than the outer circumference of the conduit at the second point.

A first distance between the first point and the second end wall may be greater than a second distance between the second point and the second end wall. The first distance may be greater than a third distance between the third point and the second end wall. The second distance may be greater than the third distance.

### BRIEF DESCRIPTION OF THE DRAWINGS

Some embodiments of the present invention are illustrated as an example and are not limited by the figures of the accompanying drawings, in which like references may indicate similar elements.

FIG. 1 is an elevation perspective view of an adapter according to an embodiment of the present invention.

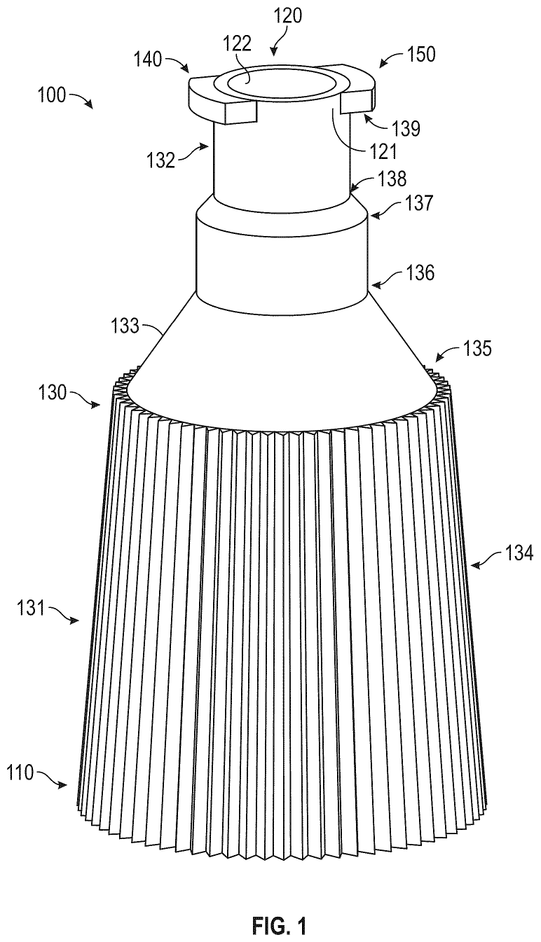

FIG. 2 is a plan perspective view of the adapter of FIG. 1.

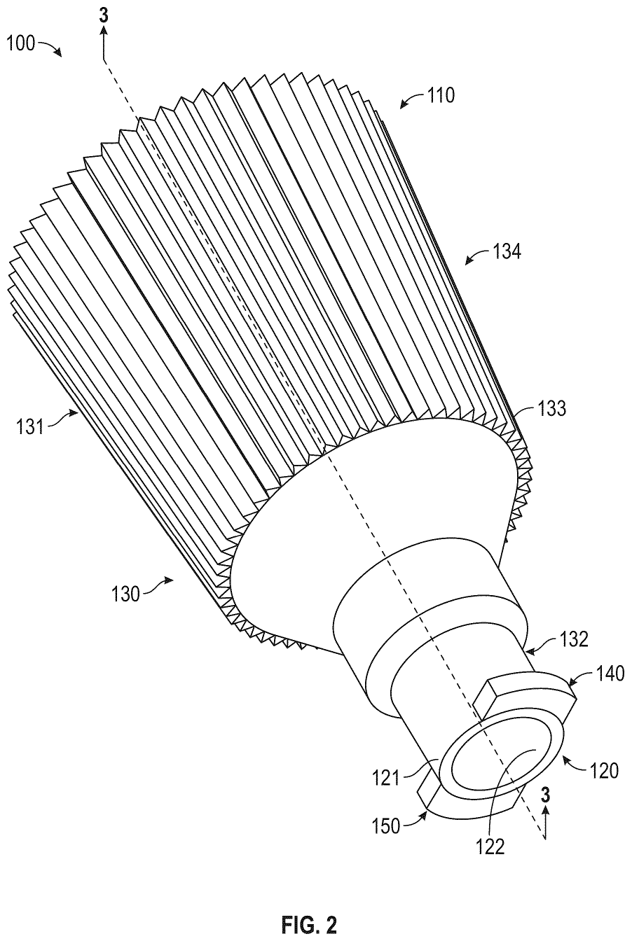

FIG. 3 is a cross-section view taken through line 3-3 of FIG. 2.

FIG. 4 is an environmental view of the adapter of FIG. 1 in combination with a syringe and a tube having contents.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

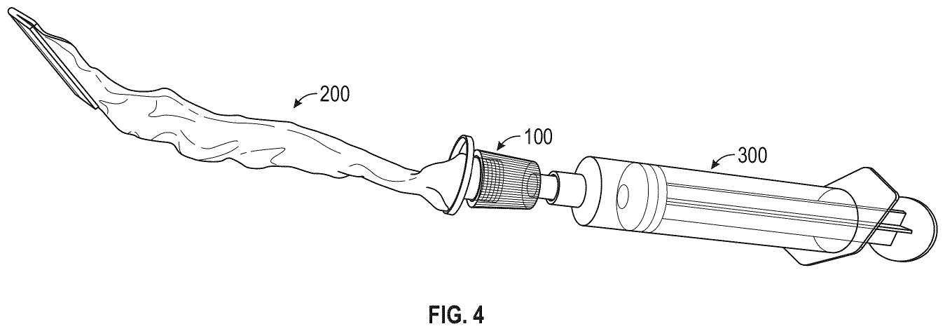

An embodiment of the invention, as shown and described by the various figures and accompanying text, provides an adapter 100 for securing a tube 200 to a syringe 300. The tube may be filled with a liquid or gel material. When the adapter 100 is secured to the tube 200 and syringe 300 as depicted in FIG. 4, the plunger of the syringe 300 may be retracted or the tube 200 may be squeezed to allow the contents of the tube 200 to enter the reservoir of the syringe 300. The adapter 100 may then be removed from the syringe 300 and the amount of the contents, now contained by the syringe 300, may be measured as they are administered by the syringe 300.

The adapter 100 may include a tube capture wall 110, a second end wall 120, a conduit 130, and a pair of protuberances 140, 150 extending from the exterior side 121 of the second end wall.

The conduit 130 may include an enclosed channel extending from a first end 131 to an opposing second end 132. The contents of the tube 200 may travel the length of the conduit 130 as they are transferred from the from the tube 200 to the syringe 300.

A tube capture wall 110 may be formed along and extend from the first end 131 of the conduit 130. Threads 111 may be formed along a surface of the tube capture wall 100. The threads 111 may be adapted to mate with the tube having contents and secure the tube to the adapter. The threads 111 may be located on an inner surface 112 of the tube capture wall 110. In such an embodiment, a threaded nozzle of the tube 200 may be contained within the tube capture wall 110 when the adapter 100 is secured to the tube 200. The threads 111 located on the tube capture wall 110 may be similar or identical to the threads located on a cap for the tube 200.

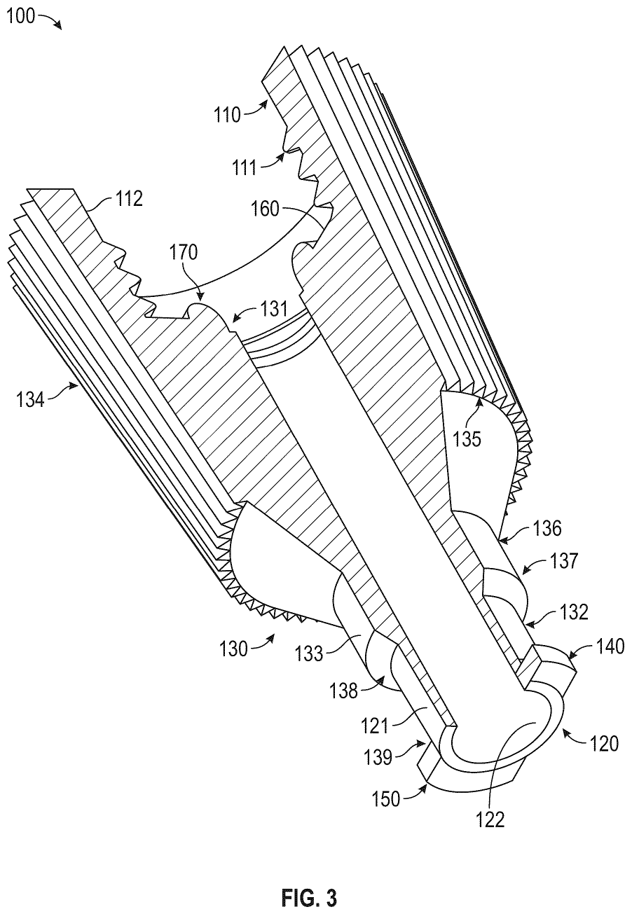

As depicted at least in FIG. 3, the tube capture wall 110 may extend away from the first end 131 of the conduit 130 and define a cylindrical volume. A first end of the cylinder may be located at a first end of the tube capture wall 110. A second end of the cylinder may be located at a second end of the tube capture wall 110, which may be proximate the first end 131 of the conduit 130. The longitudinal axis of the cylindrical volume formed by the tube capture wall 110 may be parallel to a longitudinal axis extending along the length of the conduit 130. The diameter of the cylindrical volume defined by the tube capture wall 110 may be greater than the diameter of the conduit 130. A planar surface 160 may extend between and connect the second end of the tube capture wall 110 to the first end 131 of the conduit 130. The planar surface 160 may lie in a plane perpendicular to the longitudinal axis projected along the length of the conduit 130, tube capture wall 110, and second end wall 120. An annular ridge 170 may be formed on the planar surface 160. The annular ridge 170 may extend beyond the planar surface 160 distal the conduit 130 to form a lip. The annular ridge 170 may be sized and configured to be located with the opening of the threaded nozzle of the tube 200 when the tube 200 is secured ot the adapter 100.

A second end wall 120 may be formed along and extend from the second end 132 of the conduit 130. An inner surface 122 of the second end wall 120 may define an interior circumference. The interior circumference of the second end wall 120 may be equal to an interior circumference of the conduit 130. The second end wall 120 may be monolithic with the conduit 130 and the interior surface 122 of the second end wall 120 may define the second end 132 of the conduit 130. A pair of protuberances 140, 150 may extend from the exterior surface 121 of the second end wall 120. The first protuberance 140 may be located directly opposed to the second protuberance 150 on an opposite side of the circumference of the second end wall 120. Each protuberance 140, 150 may extend perpendicularly from a longitudinal axis extending along the length of the conduit 130, second end wall 120, and tube capture wall 110. The protuberances 140, 150 may form tabs that may be captured by a luer lock syringe 300 to secure the adapter 100 to the syringe 300. The protuberances 140, 150 may be placed in the type of the syringe 300 and may then be twisted, causing the syringe 300 to capture the protuberances 140, 150 with an interference fit and retain the syringe 300 and adapter 100 in a mated configuration. The adapter 100 may be twisted in an opposite direction to disengage the syringe 300 from the adapter 100.

An exterior surface 133 of the conduit 130 may include a taper from a larger perimeter to a smaller perimeter. In one embodiment, the outer perimeter of the conduit may define a circle. In such an embodiment, the taper would be from a larger circumference to a smaller circumference. The outer circumference may taper along a portion of the conduit. The outer circumference may have a first taper at a continuous rate from the first end of the tube capture wall 110 to a first point 135 along an exterior surface 133 of the conduit 130. The outer circumference may have a second taper at a continuous rate from the first point 135 along an exterior surface 133 of the conduit 130 to a second point 136 along an exterior surface 133 of the conduit 130 or to the second end 132 of the conduit 130. The rate of the first taper may be different than the rate of the second taper.

An exterior surface 133 of the conduit 130 may include a plurality of ridges 134 extending parallel to a longitudinal axis of the conduit 130. In one embodiment, the plurality of ridges 134 may extend along an entirety of the length of the first taper from the first end of the tube capture wall 110 to the first point 131 along the exterior surface 133 of the conduit 130. The exterior surface of the second taper may not contain any ridges.

The exterior surface of the conduit 130 may have a first constant perimeter value from the second point 136 of the conduit 130, which may be the smallest diameter of the second taper, to a third point 137 along the conduit 130. The third point 137 may be closer to the second end wall 120 than the second point 136 and the second point 136 may be closer to the second end wall 120 than the first point 135. The exterior surface 133 of the conduit 130 may have a third taper at a continuous rate from third point 137 to a fourth point 138. The fourth point 138 may be closer to the second end wall 120 than the third point 137. The exterior surface of the conduit 130 may have a second constant perimeter value from the fourth point 138 to a fifth point 139, which may be smaller than the value of the first constant perimeter. The fifth point 139 may be located at the edge of the first and second protuberances 140, 150.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. An adapter for securing a tube having contents to a syringe comprising:
a conduit having a first end and an opposing second end;
a tube capture wall extending from the first end of the conduit wherein threads are located along a surface of the tube capture wall;
a second end wall having an inner surface defining the second end of the conduit and an opposing exterior surface;
a first protuberance extending from the exterior surface of the second end wall; and
a second protuberance extending from the exterior surface of the second end wall at a location opposing the first protuberance.

2. The system of claim 1 wherein the threads are located on an interior surface of the tube capture wall.

3. The system of claim 1 wherein the tube capture wall defines a cylindrical volume having a diameter greater than a diameter of the conduit.

4. The system of claim 3 wherein a longitudinal axis of the cylindrical volume is parallel to a longitudinal axis of the conduit.

5. The system of claim 4 further comprising a planar surface located between the conduit and the tube capture wall wherein the planar surface is perpendicular to the longitudinal axis of the conduit.

6. The system of claim 5 further comprising an annular ridge located on the planar surface, wherein the annular ridge lies in a plane perpendicular to the longitudinal axis of the conduit.

7. The system of claim 6 wherein the ridge protrudes from the planar surface distal the conduit.

8. The system of claim 1 wherein the second end wall is adapted to secure to a luer lock syringe.

9. The system of claim 1 wherein an exterior surface of the conduit includes a taper from a larger perimeter to a smaller perimeter.

10. The system of claim 1 wherein an exterior surface of the conduit includes a plurality of ridges extending parallel to a longitudinal axis of the conduit.

11. The system of claim 10 wherein the plurality of ridges extend continuously from a first end of the tube capture wall, which is distal the conduit, to a first point along the exterior surface of the conduit.

12. The system of claim 11 wherein an outer circumference of the conduit proximate the first point is larger than the outer circumference of the conduit at a second point along the exterior surface of the conduit.

13. The system of claim 12 wherein a first distance between the first point and the second end wall is greater than a second distance between the second point and the second end wall.

14. The system of claim 12 wherein an outer circumference of the conduit at a third point along the exterior surface of the conduit is less than the outer circumference of the conduit at the second point.

15. The system of claim 14 wherein a first distance between the first point and the second end wall is greater than a second distance between the second point and the second end wall, wherein the first distance is greater than a third distance between the third point and the second end wall, and wherein the second distance is greater than the third distance.

16. An adapter for securing a tube having contents to a syringe comprising:
a conduit having a first end, an opposing second end, and an exterior surface having a plurality of ridges extending parallel to a longitudinal axis of the conduit;
a tube capture wall extending from the first end of the conduit wherein threads are located along an interior surface of the tube capture wall;
a second end wall having an interior surface defining the second end of the conduit and an opposing exterior surface;
a first protuberance extending from the exterior surface of the second end wall; and
a second protuberance extending from the exterior surface of the second end wall at a location opposing the first protuberance; and
wherein the plurality of ridges extend continuously from a first end of the tube capture wall, which is distal the conduit, to a first point along the exterior surface of the conduit;
wherein an outer circumference of the conduit proximate the first point is larger than the outer circumference of the conduit at a second point along the exterior surface of the conduit; and
wherein a first distance between the first point and the second end wall is greater than a second distance between the second point and the second end wall.

17. The system of claim 16 wherein the second end wall is adapted to secure to a luer lock syringe.

18. The system of claim 16 wherein the tube capture wall defines a cylindrical volume having a diameter greater than a diameter of the conduit.

19. The system of claim 18 wherein a longitudinal axis of the cylindrical volume is parallel to a longitudinal axis of the conduit.

20. An adapter for securing a tube having contents to a syringe comprising:
a conduit having a first end and an opposing second end;
a tube capture wall extending from the first end of the conduit wherein threads are located along an interior surface of the tube capture wall and the tube capture wall defines a cylindrical volume having a diameter greater than a diameter of the conduit;
a second end wall having an interior surface defining the second end of the conduit and an opposing exterior surface;
a first protuberance extending from the exterior surface of the second end wall;
a second protuberance extending from the exterior surface of the second end wall at a location opposing the first protuberance;
a planar surface located between the conduit and the tube capture wall wherein the planar surface is perpendicular to the longitudinal axis of the conduit; and
an annular ridge located on the planar surface, wherein the annular ridge lies in a plane perpendicular to the longitudinal axis of the conduit and the ridge protrudes from the planar surface distal the conduit; and
wherein a longitudinal axis of the cylindrical volume is parallel to a longitudinal axis of the conduit;
wherein an exterior surface of the conduit includes a taper from a larger perimeter to a smaller perimeter;
wherein an exterior surface of the conduit includes a plurality of ridges extending parallel to a longitudinal axis of the conduit and continuously from a first end of the tube capture wall, which is distal the conduit, to a first point along the exterior surface of the conduit;
wherein an outer circumference of the conduit proximate the first point is larger than the outer circumference of the conduit at a second point along the exterior surface of the conduit and an outer circumference of the conduit at a third point along the exterior surface of the conduit is less than the outer circumference of the conduit at the second point;
wherein a first distance between the first point and the second end wall is greater than a second distance between the second point and the second end wall;
wherein the first distance is greater than a third distance between the third point and the second end wall; and
wherein the second distance is greater than the third distance.
